(19) United States
(12) Patent Application Publication
(10) Pub. No.: US 2009/0131708 A1
(43) Pub. Date: May 21, 2009

(54) Method For Production of Substituted Phenylmalonate Esters, Intermediate Compounds and The Use Thereof for production of 5, 7-dihydroxy-6-(2,4,5-trifluorophenyl)-(1,2,4)triazolo(1,5-A)pyrimidines

(75) Inventor: Norbert GOTZ, Worms (DE)

Correspondence Address: BRINKS, HOFER, GILSON & LIONE, P.O. BOX 1340, MORRISVILLE, NC, 27560, US

(73) Assignee: BASF Aktiengesellschaft, Ludwigshafen (DE)

(21) Appl. No.: 11/993,926

(22) PCT Filed: Jun. 23, 2006
(86) PCT No.: PCT/EP2006/063487

§ 371 Date: Dec. 26, 2007

(30) Foreign Application Priority Data
Jun. 27, 2005 (DE) 102005030059.6

(51) Int. Cl. C07C 69/76

(52) U.S. Cl. 560/82

(57) ABSTRACT

A process for preparing substituted phenylmalonic esters of the formula I

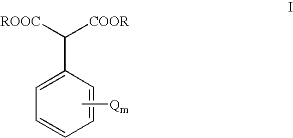

in which R is alkyl and Q is halogen, alkyl, alkoxy, haloalkyl or haloalkoxy and the index m is an integer from 1 to 5 comprising steps A) and B):
A) halogenation of compounds of the formula II,

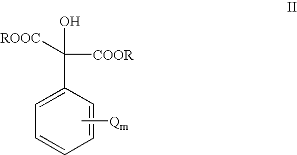

in which the variables are as defined for formula I, to give compounds of the formula III,

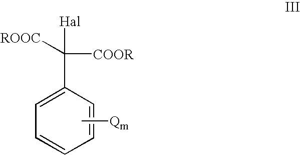

B) hydrodechlorination of the compounds of the formula III to give substituted phenylmalonic esters of the formula I;
   novel phenylmalonic ester derivatives, and also their use as intermediates.

The present invention relates to a process for preparing substituted phenylmalonic esters of the formula I

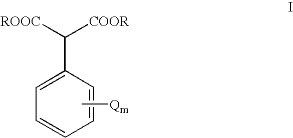

in which R is C₁-C₄-alkyl and Q is halogen, C₁-C₄-alkyl, C₁-C₄-alkoxy, C₁-C₄-haloalkyl or C₁-C₄-haloalkoxy and the index m is an integer from 1 to 5, where the groups Q can be identical or different if the index m is greater than 1, comprising steps A) and B):
A) halogenation of compounds of the formula II,

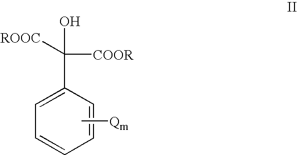

in which the variables are as defined for formula I, to give compounds of the formula III,

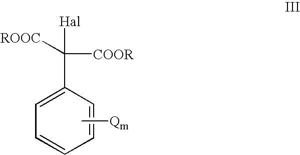

B) hydrodechlorination of the compounds of the formula III to give substituted phenylmalonic esters of the formula I.

In addition, the invention relates to novel phenylmalonic ester derivatives, and to their use as intermediates.

It was an object of the present invention to provide an economical process for preparing substituted phenylmalonic esters of the formula I, which process can be carried out on an industrial scale.

We have found that this object is achieved by the process defined at the outset.

The prior art discloses methods for preparing phenylmalonic esters; usually, they are prepared by reacting malonic esters with aryl halides in the presence of bases [cf.: U.S. Pat. No. 6,156,925; J. Org. Chem., Vol. 67, p. 541 ff (2002); Org. Lett., Vol. 4, p. 269 ff (2002); Synth. Commun. Vol. 18, p. 291 ff (1988); GB 901 880]. Phenylmalonic esters are also accessible by condensation of phenylacetic esters with dialkyl carbonates or oxalic esters [cf. Eur. J. Med. Chem., Vol. 26, p. 599 ff (1991); J. Fluorine Chem., Vol. 59, p. 225 ff (1992); Can. J. Chem., Vol. 72, p. 2312 (1994)]. These processes have the disadvantage that, for certain phenyl substitution patterns, they give only incomplete conversions, and the end products are therefore available only in very poor yields. In the process according to U.S. Pat. No. 6,156,925, Cu-containing waste waters requiring a complicated work-up produced. Accordingly, the known processes for preparing the compounds of the formula I are not fully suitable on an industrial scale.

The process according to the invention overcomes the disadvantages of the prior art. It provides an elegant access to substituted phenylmalonic esters, in particular those having one or more fluorine substituents in the phenyl ring. The process according to the invention is preferably suitable for preparing compounds I in which the index m is 1, 2, 3 or 4 and the group Q is fluorine, chlorine, methyl, methoxy, trifluoromethoxy.

Starting materials for the process according to the invention are hydroxy compounds of the formula II which can easily be obtained, for example, by coupling Grignard salts of the formula IV with mesoxalic esters of the formula V.

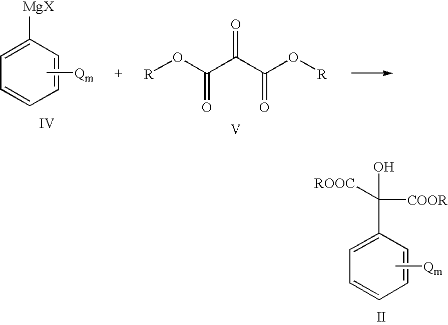

Grignard salts of the formula IV are known from the literature and can be obtained from the corresponding halobenzene derivatives, in particular bromobenzene derivatives IVA (X═Br), under generally known conditions. The Grignard reaction is usually carried out at low temperatures, preferably at temperatures of from −80° to −40° C.

Compounds of the formula II are generally known, they are prepared by reacting aromatic compounds with diethyl oxomalonate (diethyl mesoxalate) by different methods [cf.: J. Org. Chem. Vol. 47, p. 4692 ff (1982)]. Removal of the OH group can only be achieved after esterification of the OH group and subsequent reductive deoxygenation using reagents unsuitable for an industrial process, such as, for example, lithium in liquid ammonia [cf.: J. Org. Chem. Vol. 47, p. 4692 ff (1982)].

The compound of the formula IIIA, dimethyl 2-chloro-2-(4-chlorophenyl)malonate, is known. Small amounts, about 1%, of this compound are formed during the photolysis of dimethyl diazomalonate in 1,4-dichlorobenzene [cf.: Chem. Ber., Vol. 109, p. 2039 ff (1976)]. Accordingly, this method likewise provides no industrially useful access to the compounds of the formula III.

It has now been found that substituted 2-hydroxy-2-phenylmalonic esters of the formula II can be converted in a simple manner into 2-halo-2-phenylmalonic esters of the formula III, in particular 2-chloro-2-phenylmalonic esters (formula IIIA)

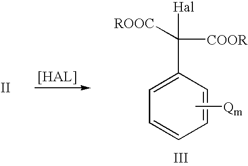

Suitable halogenating agents [HAL] are chlorinating or brominating agents, such as phosphorus oxybromide, phosphorus oxychloride, thionyl chloride, thionyl bromide or sulfuryl chloride; preference is given to using phosphorus halides, such as POCl₃, PCl₅, POBr₃ or PBr₅, in particular POCl₃/PCl₅ or POBr₃/PBr₅ systems. The reaction can be carried out in the absence or presence of a solvent. Customary reaction temperatures are from 0 to 150° C. or, preferably, from 80 to 125° C.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether (MTBE), dioxane, anisole and tetrahydrofuran (THF), ketones, such as acetone, methyl ethyl ketone, diethyl ketone and tert-butyl methyl ketone, particularly preferably in the halogenating agent or halogenated hydrocarbons. It is also possible to use mixtures of the solvents mentioned.

The starting materials are generally reacted with one another in equimolar amounts. In terms of yield, it may be advantageous to employ an excess of [HAL], based on II.

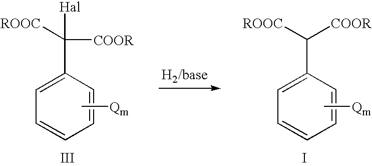

The dehalogenation of the compounds of the formula III and the conversion into the phenylmalonic esters is carried out by catalytic hydrogenation. This reaction is usually carried out at temperatures of from 0° C. to 150° C., preferably from 10° C. to 100° C., in an inert organic solvent in the presence of a base [cf. JP 04224535].

Suitable solvents are water, alcohols, such as methanol, ethanol, n-propanol, iso-propanol, n-butanol and tert-butanol, and also dimethyl sulfoxide, dimethylformamide and dimethylacetamide or carboxylic acids. Preferred solvents are ethers, such as THF, dioxane and alcohols. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide, potassium amide, alkali metal and alkaline earth metal carbonates, such as lithium carbonate, potassium carbonate and calcium carbonate, and also alkali metal bicarbonates, such as sodium bicarbonate, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to tertiary amines.

The bases are generally employed in equimolar amounts, in excess or, if appropriate, as solvent. Preference is given to using 3-4 mol equivalents, based on the compound of the formula III.

The reaction is preferably carried out in the presence of a catalyst, such as transition metal catalysts, in particular nickel, cobalt, palladium, platinum, ruthenium, rhodium or copper catalysts. Preference is given to Pd/C, Pt/C and Raney-Ni or mixtures thereof.

The introduction of hydrogen into the reaction mixture is preferably carried out at temperatures between 0° and +50° C., at atmospheric pressure or at a superatmospheric pressure of up to about 10 bar.

Work-up and purification of the reaction products is preferably carried out by distillation. The individual products can be identified both by HPLC and GC analysis.

The phenylmalonic esters, easily obtainable by the process according to the invention, are suitable as intermediates for preparing dyes or active compounds in the pharmaceutical or agrochemical field. In the preparation of active [1,2,4]triazolo[1,5-a]pyrimidine compounds, they are reacted with 3-amino-1,2,4-triazole to give 5,7-dihydroxy-6-phenyl[1,2,4]triazolo[1,5-a]pyrimidines [cf. EP-A 550 113, EP-A 975 634, U.S. Pat. No. 5,808,066, U.S. Pat. No. 6,117,876, WO 98/46607].

### PROCESS EXAMPLES

### Example 1

### Preparation of diethyl 2-hydroxy-2-(2′,4′,6′-trifluorophenyl)malonate

At 20 to 25° C., 12.5 ml of a 2M isopropylmagnesium chloride solution in tetrahydrofuran (THF) were added to a mixture of 5.4 g of 2,4,6-trifluorobromobenzene in 30 ml of THF, resulting in an exothermic reaction, and the temperature increased to 54° C. This solution was cooled to −55° C., and a solution of 17.4 g of diethyl ketonemalonate (diethyl mesoxalate) in 10 ml of THF was added dropwise. After a further 40 min at −55° C., first 12.5 ml of water and then 12.5 ml of 10% strength hydrochloric acid were, at 0° C., added to the reaction mixture. The aqueous phase was saturated with Na₂SO₄, the phases were separated, the aqueous phase was extracted with THF, the solvent of the combined organic phases was removed and the residue was distilled at 0.4 mbar. This gave 6.7 g of the title compound of b.p. 158-160° C./7 mbar. This corresponds to a yield of 88% of theory.

### Example 2

### Preparation of diethyl 2-chloro-2-(2′,4′,6′-trifluorophenyl)malonate

### Step A

At 25° C., a solution of 13.5 g of diethyl 2-hydroxy-2-(2′,4′,6′-trifluorophenyl)malonate in 75 ml of POCl₃ was reacted with 14.9 g of phosphorus pentachloride. After the reaction had ended, the crude product obtained was distilled at 0.25 mbar. This gave 13.8 g of the title compound of b.p. 143-144° C./2 mbar. This corresponds to a yield of 97% of theory.

### Example 3

### Preparation of diethyl 2,4,6-trifluorophenylmalonate

### Step B

5.0 g of Raney nickel were suspended in a solution of 11.8 g of diethyl 2-chloro-2-(2′,4′,6′-trifluorophenyl)malonate and 3.5 g of triethylamine in 300 ml of THF. At about 24° C., at a slight superatmospheric pressure, 700 ml of hydrogen were introduced into this suspension over a period of 135 min. The reaction product was filtered through kieselguhr and the solvent was then removed from the filtrate.

This gave 10.4 g of the title compound of b.p. 125° C./5 mbar. This corresponds to a yield of 99.2% of theory.

1-11. (canceled)

12. A process for preparing a substituted phenylmalonic ester of formula I: 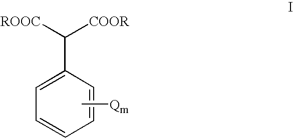
comprising the steps of:
A) halogenation of a compound of formula II: 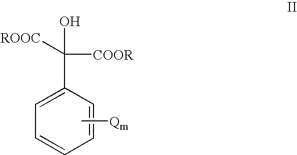
resulting in a compound of formula III: 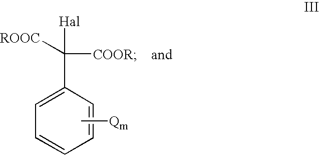
B) hydrodechlorination of the compound of formula III, wherein the substituted phenylmalonic ester of formula I is prepared;
wherein R is C₁-C₄-alkyl and Q is halogen, C₁-C₄-alkyl, C₁-C₄-alkoxy, C₁-C₄-haloalkyl or C₁-C₄-haloalkoxy; and m is an integer from 1 to 5; wherein the groups Q can be identical or different if m is greater than 1.

13. The process of claim 12, wherein step A uses POCl₃, PCl₅, POBr₃ and/or PBr₅.

14. The process of claim 12, wherein step B is carried out in the presence of a catalyst and an amine.

15. The process of claim 13, wherein step B is carried out in the presence of a catalyst and an amine.

16. The process of claim 14, wherein the catalyst is selected from the group consisting of a nickel catalyst, a cobalt catalyst, a palladium catalyst, a platinum catalyst, a ruthenium catalyst, a rhodium catalyst and a copper catalyst.

17. The process of claim 15, wherein the catalyst is selected from the group consisting of a nickel catalyst, a cobalt catalyst, a palladium catalyst, a platinum catalyst, a ruthenium catalyst, a rhodium catalyst and a copper catalyst.

18. The process of claim 14, wherein a tertiary amine is used.

19. The process of claim 15, wherein a tertiary amine is used.

20. The process of claim 16, wherein a tertiary amine is used.

21. The process of claim 17, wherein a tertiary amine is used.

22. The process of claim 12, wherein at least one group Q in the formulae I, II and III is a fluorine atom.

23. The process of claim 12, wherein m is 3, Q is fluoro, and Qₘ in the formulae I, II and III is 2,4,6-trifluoro.

24. The process of claim 12, further comprising:
reacting a salt of the formula IV: 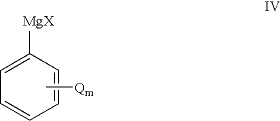
with a mesoxalic ester of formula V: 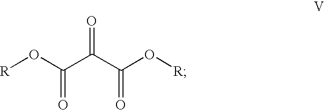
wherein X is chlorine or bromine, and Qₘ and R are as defined for claim 12;
wherein the compound of formula II is prepared prior to step A.

25. The process of claim 24, wherein at least one group Q in the formulae I, II and III is a fluorine atom.

26. The process of claim 24, wherein m is 3, Q is fluoro, and Qₘ in the formulae I, II and III is 2,4,6-trifluoro.

27. A process for preparing a 5,7-dihydroxy-6-phenyl[1,2,4]triazolo[1,5-a]pyrimidine comprising:
reacting a substituted phenylmalonic ester of formula I: 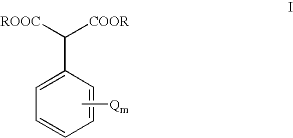
with 2-amino-1,3,5-triazole;
wherein R is C₁-C₄-alkyl and Q is halogen, C₁-C₄-alkyl, C₁-C₄-alkoxy, C₁-C₄-haloalkyl or C₁-C₄-haloalkoxy; and m is an integer from 1 to 5; wherein the groups Q can be identical or different if m is greater than 1;
wherein a 5,7-dihydroxy-6-phenyl[1,2,4]triazolo[1,5-a]pyrimidine is prepared.

28. The process of claim 27, wherein at least one group Q in the formulae I, II and III is a fluorine atom.

29. The process of claim 27, wherein m is 3, Q is fluoro, and Qₘ in the formulae I, II and III is 2,4,6-trifluoro.

30. A compound of formula IIIA: 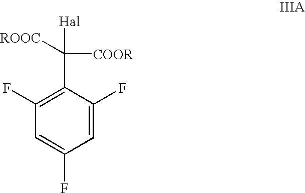
wherein R is C₁-C₄-alkyl.
